(19) United States
(12) Patent Application Publication
(10) Pub. No.: US 2014/0186889 A1
(43) Pub. Date: Jul. 3, 2014

(54) ANTIBODY GENERATION FROM PLASMACYTOMA-PRONE TRANSGENIC ANIMALS

(71) Applicant: Richard A. Shimkets, Commerce, GA (US)

(72) Inventor: Richard A. Shimkets, Commerce, GA (US)

(73) Assignee: Abeome Corporation, Athens, GA (US)

(21) Appl. No.: 14/111,978

(22) PCT Filed: Apr. 16, 2012
(86) PCT No.: PCT/US12/33797

§ 371 Date: Jan. 29, 2014

Related U.S. Application Data
(60) Provisional application No. 61/475,950, filed on Apr. 15, 2011.

(51) Int. Cl. C07K 16/00

(52) U.S. Cl. CPC C07K 16/00
USPC 435/69.6; 435/70.4; 435/326

(57) ABSTRACT

Certain transgenic animals which are prone to the rapid cell division of their antibody-secreting cells have superior properties for the generation of monoclonal antibodies. Not only can their antibody producing cells can be made into hybridomas with superior growth to hybridomas from non-prone animals, but the antibody producing cells themselves can be cultured directly without cell fusion or further manipulation. Disclosed herein are methods of making monoclonal antibodies comprising exposing the transgenic animals disclosed herein to an antigen and extracting antigen-specific antibody secreting cells from the transgenic animal.

This application claims the benefit of U.S. Provisional Application No. 61/475,950, filed on Apr. 15, 2011 which is incorporated herein by reference in its entirety.

### I. BACKGROUND

1. Kohler and Milstein's (1975) seminal publication describes how monoclonal antibodies can be obtained by the generation of hybridoma cells, heterokaryons resulting from the fusion of mouse B-lymphocytes, and immortal myeloma cells. In this process, an animal is immunized with an antigen for which one desires antibodies to be made, and the antibody-producing cells are harvested and typically fused to an immortal myeloma cell. The relevant hybridoma cells producing the antibody of choice are separated into individual clones using Limiting Dilution Subcloning (LDS) or a similar technology. Each individual cell population testing positive for the target must be processed by reiterative cycles of LDS until the progeny of a positive cell is mathematically identified as clonal (Staszewski, 1984). Recovering the hybridomas using LDS cell cloning is perhaps the most problematic, time consuming, and labor-intensive step in generating mAbs (Antczak, 1982; O'Reilly et al., 1998). The LDS process is eliminated altogether when all the desired hybridoma cells, expressing the membrane immunoglobulin isovariant of the secreted antibody, are identified and deposited individually (cloned) into culture plates using Fluorescence Activated Cell Sorting (FACS): the DiSH protocol. Abeome successfully addressed FACS cloning by engineering hybridomas that efficiently express membrane immunoglobulin (the BCR complex) and allow the Direct Selection of Hybridomas (DiSH) (Price et al., 2009).

2. Plasmacytes represent a terminally-differentiated population of B cells whose role is the secretion of antibodies in response to immunologic insult. It has been proposed that plasmacytes represent the major population of cells that results in successful hybridoma fusions (Paslay and Roozen, 1981). However, all cell fusion-based technologies are inefficient and prevent effective sampling of the estimated 10³ to 10⁵ specifically reactive plasmacytes from an immunized animal (Han et al., 2003). The fusion step creating a hybridoma from a plasmacyte and an immortal cell, typically a myeloma, is a very inefficient step, with an estimated loss of 99.9% of plasmacytes. What is needed are animals that produce more antibody-secreting plasmacytes, or plasmacytes that can be grown in culture from a single clonal cell without the need for fusion so that many distinct antibodies can be generated in parallel. Many attempts to do this have been undertaken, ranging from viral transformation technologies to conditional oncogenes.

### II. SUMMARY

3. Disclosed are methods related to making one or more monoclonal antibodies wherein an animal strain prone to develop plasmacyte hyperplasia or plasmacytoma is immunized with an antigen of interest, and antibody producing cells are extracted.

4. In one aspect the disclosed methods utilize IL-6 transgenic animals.

5. Also disclosed herein are hybridomas made by the disclosed methods.

### III. DETAILED DESCRIPTION

6. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

7. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

8. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

9. In this specification and in the claims which follow, reference will be made to a number of terms which shall be defined to have the following meanings:

10. “Optional” or “optionally” means that the subsequently described event or circumstance may or may not occur, and that the description includes instances where said event or circumstance occurs and instances where it does not.

11. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

12. The methods disclosed herein relate to the production of monoclonal antibodies. Typically, prior to the present disclosure, to produce an antibody of interest, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. Alternatively, monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). The present disclosure changes this method.

13. In one aspect, disclosed herein are methods of making one or more monoclonal antibodies comprising immunizing an animal strain prone to develop plasmacyte hyperplasia or plasmacytoma with an antigen of interest and extracting antibody producing cells. Once antibody producing cells are extracted, one or more monoclonal antibodies can be made from the antibody producing cells. It is understood and contemplated herein that the antibody producing cells from the prone mice can be cultured directly or immortalized by cellular fusion. In one aspect, an antibody producing cell prone animal can be an animal with a transgene that results in antibody producing cell production. For example, the animal can be a mammalian animal containing a transgene for the over-expression of a mammalian interleukin-6 (IL-6) gene, such as the human, bovine, porcine, equine, rat, guinea pig, feline, canine, rabbit, non-human primate, or murine interleukin 6 gene. Thus, in one aspect disclosed herein are methods of making one or more monoclonal antibodies comprising immunizing an animal containing a transgene for a mammalian IL-6 with an antigen of interest and extracting antibody producing cells. In a further aspect, the mammalian IL-6 gene can be over murine or human origin.

14. Antibodies are typically proteins which exhibit binding specificity to a specific antigen. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain can have regularly spaced intrachain disulfide bridges. Each heavy chain can have at one end a variable domain (V(H)) followed by a number of constant domains. Each light chain can have a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There currently are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The present invention provides the presentation of all of the immunoglobulin classes via binding to Ig α and/or Ig β. The heavy chain constant domains that correspond to the different classes of immunoglobulins are called alpha, delta, epsilon, gamma, and mu, respectively.

15. The Immunoglobulin (Ig) heavy chain genes are typically complex transcription units with multiple poly(A) sites in which changes in the cleavage and polyadenylation machinery can play an important role in B-cell, stage-specific expression. Ig μ heavy chains can be expressed in pre, immature, and mature-B-cells and IgM+ plasma cells. The α, ε, and γ heavy chains can be expressed in memory and IgA+, IgE+, and IgG+ plasma cells, respectively (Janeway and Travers, 1994). RNA from each of the five classes of Ig heavy chain genes (α, δ, ε, γ, μ) can be alternatively processed to produce two types of mRNAs: one encodes the secreted form of the Ig protein and is produced by use of the promoter-proximal, weak Ig sec (secretory-specific) poly(A) site in plasma cells; the other mRNA encodes the membrane-bound (mb) receptor for antigen on the surface of mature or memory B-cells and can be produced by use of the downstream, strong Ig membrane poly(A) site [Alt, 1980; Rogers, 1980; Rogers, 1981].

16. There can be a 2-5-fold change in the transcription rate of the Ig genes in different B-cell stages (Kelly and Perry, 1986). The site of termination can vary in the μ (Galli et al., 1987; Guise et al., 1988; Yuan and Tucker, 1984) but not the γ and α genes (Flaspohler et al., 1995; Flaspohler and Milcarek., 1990; Lebman et al., 1992). RNA processing events can play the major role in determining the ratios of the two forms of IgG heavy chain mRNA as first shown in 1985 (Milcarek and Hall, 1985). The crucial role for RNA processing has been further substantiated (See Edwalds-Gilbert and Milcarek, 1995; Edwalds-Gilbert and Milcarek, 1995; Flaspohler et al., 1995; Flaspohler and Milcarek., 1990; Genovese et al., 1991; Genovese and Milcarek, 1990; Hall and Milcarek, 1989; Kobrin et al., 1986; Lassman et al., 1992; Lassman and Milcarek, 1992; Matis et al., 1996; Milcarek et al., 1996). See also (Edwalds-Gilbert et al., 1997). Polyadenylation at the weak secretory-specific poly(A) site, which is promoter proximal to the membrane specific poly(A) site, and splicing to the membrane-specific exons at the sub-optimal splice site, in the last secretory-specific exon, can bemutually exclusive events. It has been shown that changes in the cleavage and polyadenylation of the precursor RNA tip the balance in plasma cells to the use of the first, weak poly(A) site.

17. The term “variable” is used herein to describe certain portions of the variable domains which differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains can each comprise four FR regions, largely adopting a β-sheet configuration, connected by three CDRs, which form loops connecting, and in some cases forming part of, the -sheet structure. The CDRs in each chain can be held together in close proximity by the FR regions and, with the CDRs from the other chain, contribute to the formation of the antigen binding site of antibodies (see Kabat E. A. et al., “Sequences of Proteins of Immunological Interest” National Institutes of Health, Bethesda, Md. (1987)). The constant domains are not typically involved directly in binding an antibody to an antigen, but exhibit various effector functions, such as participation of the antibody in antibody-dependent cellular toxicity.

18. As used herein, “monoclonal antibody” refers to an antibody that is produced by cells that are all derived from a single antibody-producing cell type and has a specific affinity for an antigen. Monoclonal antibodies are obtained from a substantially homogeneous population of antibodies, i.e., the individual antibodies comprising the population are identical except for possible naturally occurring mutations that may be present in minor amounts. The monoclonal antibodies secreted by the hybridoma cells of the present invention can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

19. It is understood that the transgenic animal can be a rat, mouse, pig, cow, horse, rabbit, dog, cat, or non-human primate.

20. In one aspect disclosed herein are hybridoma cells made by the disclosed methods. Thus, in one aspect, disclosed herein are hybridomas made by immunizing an animal prone to develop plasmacyte hyperplasia or plasmacytoma with an antigen of interest, extracting the antibody producing cells, and fusing the antibody producing cell with an immortalized cell. Alternatively immortalization of the antibody producing cells can take place by transfection of the antibody producing cells with exogenous DNA. As used herein, “hybridoma” is a cell or a cell line that is produced by fusing an antibody producing cell, e.g. a B cell, and an immortalized cell, e.g. a myeloma cell. As used herein “B cell” means an immature B cell, a mature naïve B cell, a mature activated B cell, a memory B cell, a B lineage lymphocyte, a plasma cell or any other B lineage cell of human origin or from non-human animal sources. The hybridomas of this invention can be made by fusing a B cell of human origin or from non-human animal sources, with an immortalized cell line using a suitable fusing agent, such as polyethylene glycol, to form a hybridoma cell (Goding, “Monoclonal Antibodies: Principles and Practice” Academic Press, (1986) pp. 59-103).

21. In order to obtain the B cells for the production of a hybridoma, a mouse or other appropriate host animal, is typically immunized with an immunizing agent or antigen to elicit B cells that produce or are capable of producing antibodies that will specifically bind to the immunizing agent or antigen. Alternatively, the B cells may be immunized in vitro. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, although HAT is not necessary for DISH, typically, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

22. Preferred immortalized cell lines are those that fuse efficiently, support high level expression of antibody by the selected antibody-producing cells, and are sensitive to a medium. The immortalized cell line can be sensitive to HAT medium. More preferred immortalized cell lines are murine myeloma lines, which can be obtained, for instance, from the Salk Institute Cell Distribution Center, San Diego, Calif. and the American Type Culture Collection (ATCC), Rockville, Md. Human myeloma and mouse-human heteromyeloma cell lines also have been described for the production of human monoclonal antibodies (Kozbor, J. Immunol., 133:3001 (1984) and Brodeur et al., “Monoclonal Antibody Production Techniques and Applications” Marcel Dekker, Inc., New York, (1987) pp. 51-63). For example, the following myeloma cell lines can be obtained from the ATCC: MOPC-31C, RPMI 8226, IM-9, MPC-11, CCL-189, HK-PEG-1, HS-Sultan, A2B5 clone 105, P3X63Ag8.653, Sp2/0-Ag14, Sp2/0-Ag14/SF, P3X63Ag8U.1, HFN 36.3 HFN 7.1, 45.6.TG1.7, ARH-77, Y3-Ag 1.2.3, SJK-132-20, SJK-287-38 and SJK-237-71.

23. The hybridoma cells of the present invention can be assayed for surface expression and the culture medium in which the hybridoma cells are cultured can be assayed for the presence of monoclonal antibodies directed against a desired immunogen by methods known in the art such as ELISA, western blot, FACS, magnetic separation etc. The binding specificity of monoclonal antibodies secreted by the hybridoma cells can be, for example, determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson et al., Anal. Biochem., 107:220 (1980).

24. After a desired hybridoma cell is identified, either by assaying surface expression or by assaying the culture medium, the selected hybridoma cell can be grown by standard methods. Suitable culture media for this purpose include, for example, Dulbecco's Modified Eagle's Medium and RPMI-1640 medium. Alternatively, the hybridoma cells may be grown in vivo as ascites in a mammal.

25. As used herein, “a population of hybridoma cells” means a sufficient number of cells such that a percentage of the cells expressing antibody can be determined. The hybridoma cells of the population can be cells from a pure hybridoma cell line where all of the cells of the line produce only one monoclonal antibody specific for a particular antigen or a mixture of cells wherein multiple monoclonal antibodies are produced. Thus, a population of hybridoma cells can produce more than one monoclonal antibody such that some cells produce a monoclonal antibody that recognize one antigen and other cells in the population produce monoclonal antibody that recognizes a second antigen and other cells in the population produce a monoclonal antibody that recognizes a third antigen etc.

26. As used herein, “express” means that the monoclonal antibody can be detected by means standard in the art such as Western blot, ELISA, immunofluorescence, hemolytic assay, fluorescence activated cell sorting (FACS) as they are currently practiced in the art.

27. Once hybridomas are isolated by the present invention, the antibody coding regions of the hybridomas can be used to make monoclonal antibodies by recombinant DNA methods, such as those described in U.S. Pat. No. 4,816,567 or U.S. Pat. No. 6,331,415. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody comprising one antigen-combining site having specificity for one antigen and second antigen-combining site having specificity for a different antigen.

28. Thus in one aspect, the present invention also contemplates hybridomas made from plasmacytoma or plasmacyte hyperplasia-prone animals. As defined herein, a “plasmacytoma” is a discrete, solitary mass of neoplastic monoclonal plasma cells (plasmacytes) in either bone or soft tissue (extramedullary). The types of plasmacytomas include but are not limited to Soft-tissue or nonosseous extramedullary plasmacytoma (EMP); Solitary bone plasmacytoma (SBP); Multifocal form of multiple myeloma; Multiple myeloma; and Plasmablastic sarcoma. In animal models, prior to the development of plasmacytoma there is often a significant amount of plasmacyte hyperplasia, a condition in which plasmacytes are rapidly dividing and present in excess. This can cause a significant increase in cell numbers and, for example, can lead to an enlarged spleen.

B. Examples

29. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

30. Cells have been cultured successfully from certain spontaneous plasmacytomas for many generations, in experiments dating back more than 40 years (Astaldi et al., 1968). New genetic models of plasmacytoma exist which have not been studied previously for uses of monoclonal antibody generation. Interleukin-6 transgenic mice (IL6-mice) (Kovalchuk et al., 2002) display significant plasmacyte hyperplasia and a high frequency of plasmacytomas. Spleens are enlarged as much as 20-fold in these models. The tumor formation phenotype is dependent on a translocation event of the myc and IgH genes, which causes overexpression of myc in plasmacytes due to regulation by the IgH promoter. A similar event and phenotype results from Bc12 transgenic mice (Silva et al., 2003). The translocation events that happen with significant frequency in the IL6 and Bc12 mice have been studied extensively, and three lines of mice have been established with insertion sequences that mimic the three most common translocations (Park et al., 2005, Cheung et al., 2004). All 5 of the plasmacytoma-prone transgenic lines above result in rapid onset of plasma cell hyperplasia and plasmacytoma formation when treated with pristane, which provides for the creation of a pristane-inducible system of generating a large percentage of culturable cells.

31. Herein, it was determined whether the antibody-producing cells of any of these plasmacytoma-prone transgenic mice can be more efficiently immortalized by cell fusion methods to produce hybridomas, or isolated and cultured directly. This was done by isolating CD220-, CD138+plasmacytes from the spleens of transgenic mice using Magnetic Activated Cell Separation (MACS). Cells were plated and screened for growth and antibody production.

32. All of transgenes confer some ability to grow in culture, but the most successful growing cells in both longevity in culture and in antibody secretion were those from the BCL2 and IL6 mice (Table 1)

TABLE 1
Plasmacytoma-Prone Mouse Strains and Survival of their Cells in Culture

| | | Time To | | |
|---|---|---|---|---|
| | | Tumor | | |
| | | Development | Survival | |
| Strain | Zygosity | (% of Animals) | in Culture | References |
| C.TV1 | Hetero | 21 months | 60 Days | Park |
| (C.iMycEμ) | | (68%) | | |
| C.TV2 | Homo | Not Yet | 60 Days | S. Janz-Personal |
| (iMycCμ) | | Determined | | Communication |
| C.TV3 | Hetero | 380 days | 60 Days | Cheung |
| (C.iMycCα) | | (9.3%) | | |
| C.Bcl2 | Hetero | 113 days (56%) | >4 Months | Silva |
| C.IL6 | Hetero | 12 months | >6 Months | Kovalchuk |
| | | (40%) | | |
| C. | None | No Tumors | 1 Month | |
| (Wild type) | | | | |

33. The cultured plasmacytes from the Bc12 and IL6 mice secrete all antibody isotypes tested (Table 2), and have different growth morphologies and rates. The genetic cross between both mice, generating a Bc12/IL6 mouse, had growth properties equivalent to the IL6 mice alone.

TABLE 2
Relative quantities of antibodies secreted into culture media
from cells isolated from various transgenic mouse strains

| Mouse | IgM | IgA | IgG1 | IgG2a | IgG2b | IgG3 | κ | λ |
|---|---|---|---|---|---|---|---|---|
| C. BCL2 | 1.284 | 0.412 | 1.584 | 1.210 | 1.288 | 0.533 | 1.451 | 0.972 |
| C. IL6 | 1.146 | 0.549 | 1.155 | 0.516 | 0.498 | 0.774 | 1.222 | 0.409 |
| C. TV1/ | 1.038 | 0.161 | 0.320 | 0.051 | 0.332 | 0.174 | 0.695 | 0.110 |
| IL6 | | | | | | | | |
| B6 TV | 0.042 | 0.031 | 0.032 | 0.027 | 0.029 | 0.034 | 0.036 | 0.038 |
| C. TV3 | 0.542 | 0.127 | 0.034 | 0.039 | 0.130 | 0.036 | 0.418 | 0.087 |

This novel discovery makes mice bearing these transgenes very useful for the generation of monoclonal antibodies, because traditional cell fusion hybridoma technology will be replaced by directly culturing cells from these mice. This can be further improved by standard viral immortalization methods which typically fail on senescent plasmacytes.

34. Additionally, these mice can be crossed with other mice, such as Abeome's transgenic mice bearing Ig-alpha or Ig-alpha and Ig-beta of the B cell receptor complex, yielding mice whose plasmacytes can be cultured AND can be selected for directly by virtue of their cell-surface antibody. Likewise, crossing these mice with transgenic mice that produce human antibodies can significantly improve the yield of recoverable antibodies in those mice.

35. Additionally, since these transgenic animals are overproducing plasmacytes, significant numbers of antibody-producing cells can be directly isolated from non-terminal bleeds, so that a single animal can be used for multiple experiments.

36. Thus, the present claims and disclosure herein stand on their own as novel methods of isolating cells that can grow in culture and that produce antibody, or in combination with other transgenic animals can make other transgenic technologies more useful.

C. References

Antczak, D. F. (1982). Monoclonal antibodies: technology and potential use. J Am Vet Med Assoc 181, 1005-10.
Astaldi, G., Eridani, S., and Ponti, G. B. (1968). The proliferative activity of plasma cells from plasmocytoma in vitro. Eur J Cancer 4, 9-13.
Cheung, W. C., Kim, J. S., Linden, M., Peng, L., Van Ness, B., Polakiewicz, R. D., and Janz, S. (2004). Novel targeted deregulation of c-Myc cooperates with Bc1-X(L) to cause plasma cell neoplasms in mice. J Clin Invest. 113, 1763-73.
Harris J F, et al. Increased frequency of both total and specific monoclonal antibody producing hybridomas using a fusion partner that constitutively expresses recombinant IL-6. J. Immunol. Methods 148: 199-207, 1992. PubMed: 1373425
Kohler, G., and Milstein, C. (1975). Continuous cultures of fused cells secreting antibody of predefined specificity. J Immunol 174, 2453-5.
Kovalchuk, A. L., Kim, J. S., Park, S. S., Coleman, A. E., Ward, J. M., Morse, H. C., 3rd, Kishimoto, T., Potter, M., and Janz, S. (2002). IL-6 transgenic mouse model for extraosseous plasmacytoma. Proc Natl Acad Sci U S A 99, 1509-14.
Meagher, R. B. (2006b). Method of Rapid Production of Hybridomas Expressing Monoclonal Antibodies on the Cell Surface, U.S. Pat. No. 7,148,040
Meagher, R. B. (2009). Genetically altered hybridomas, myelomas and B cells that facilitate the rapid production of monoclonal antibodies, U.S. Pat. No. 7,629,171
O'Reilly, L. A., Cullen, L., Moriishi, K., O'Connor, L., Huang, D. C., and Strasser, A. (1998). Rapid hybridoma screening method for the identification of monoclonal antibodies to low-abundance cytoplasmic proteins. Biotechniques 25, 824-30.
Park, S. S., Shaffer, A. L., Kim, J. S., duBois, W., Potter, M., Staudt, L. M., and Janz, S. (2005). Insertion of Myc into Igh accelerates peritoneal plasmacytomas in mice. Cancer Res. 65(17):7644-52.
Paslay, J., and Roozen, K. (1981). The effect of B cell stimulation on hybridoma formation, In Monoclonal antibodies and T cell Hybridomas: Perspectives and Technical Advances (G. J. Hammerling and J. F. Kearney, eds.), Elsevier, New York.
Price, P. W., McKinney, E. C., Wang, Y., Sasser, L. E., Kandasamy, M. K., Matsuuchi, L., Milcarek, C., Deal, R. B., Culver, D. G., and Meagher, R. B. (2009). Engineered cell surface expression of membrane immunoglobulin as a means to identify monoclonal antibody-secreting hybridomas. J Immunol Methods 343, 28-41.
Silva, S., Kovalchuck, A. L., Kim, J. S., Klein, G and Janz, S. (2003). BLC2 Accelerates Inflammation-induced BALB/c Plasmacytomas and Promotes Novel Tumors with Coexisting T(12;15) and T(6;15) Translocations. Cancer Res. 63, 8656-8663.
Staszewski, R. (1984). Cloning by limiting dilution: an improved estimate that an interesting culture is monoclonal. Yale J Biol Med 57, 865-8.

D. Sequences
SEQ ID NO: 1
Human IL-6 mRNA (Accession NO. NM_000600.3)
AATATTAGAGTCTCAACCCCCAATAAATATAGGACTGGAGATGTCTGAGGCTCATTCTGCCCTCGAGCCC
ACCGGGAACGAAAGAGAAGCTCTATCTCCCCTCCAGGAGCCCAGCTATGAACTCCTTCTCCACAAGCGCC
TTCGGTCCAGTTGCCTTCTCCCTGGGGCTGCTCCTGGTGTTGCCTGCTGCCTTCCCTGCCCCAGTACCCC
CAGGAGAAGATTCCAAAGATGTAGCCGCCCCACACAGACAGCCACTCACCTCTTCAGAACGAATTGACAA
ACAAATTCGGTACATCCTCGACGGCATCTCAGCCCTGAGAAAGGAGACATGTAACAAGAGTAACATGTGT
GAAAGCAGCAAAGAGGCACTGGCAGAAAACAACCTGAACCTTCCAAAGATGGCTGAAAAAGATGGATGCT
TCCAATCTGGATTCAATGAGGAGACTTGCCTGGTGAAAATCATCACTGGTCTTTTGGAGTTTGAGGTATA
CCTAGAGTACCTCCAGAACAGATTTGAGAGTAGTGAGGAACAAGCCAGAGCTGTGCAGATGAGTACAAAA
GTCCTGATCCAGTTCCTGCAGAAAAAGGCAAAGAATCTAGATGCAATAACCACCCCTGACCCAACCACAA
ATGCCAGCCTGCTGACGAAGCTGCAGGCACAGAACCAGTGGCTGCAGGACATGACAACTCATCTCATTCT
GCGCAGCTTTAAGGAGTTCCTGCAGTCCAGCCTGAGGGCTCTTCGGCAAATGTAGCATGGGCACCTCAGA
TTGTTGTTGTTAATGGGCATTCCTTCTTCTGGTCAGAAACCTGTCCACTGGGCACAGAACTTATGTTGTT
CTCTATGGAGAACTAAAAGTATGAGCGTTAGGACACTATTTTAATTATTTTTAATTTATTAATATTTAAA
TATGTGAAGCTGAGTTAATTTATGTAAGTCATATTTATATTTTTAAGAAGTACCACTTGAAACATTTTAT
GTATTAGTTTTGAAATAATAATGGAAAGTGGCTATGCAGTTTGAATATCCTTTGTTTCAGAGCCAGATCA
TTTCTTGGAAAGTGTAGGCTTACCTCAAATAAATGGCTAACTTATACATATTTTTAAAGAAATATTTATA
TTGTATTTATATAATGTATAAATGGTTTTTATACCAATAAATGGCATTTTAAAAAATTCAGCAAAAAAAA
AAAAAAAAAAA
SEQ ID NO: 2
Murine IL-6 mRNA (Accession NO. NM_031168.1)
CCAAGAACGATAGTCAATTCCAGAAACCGCTATGAAGTTCCTCTCTGCAAGAGACTTCCATCCAGTTGCC
TTCTTGGGACTGATGCTGGTGACAACCACGGCCTTCCCTACTTCACAAGTCCGGAGAGGAGACTTCACAG
AGGATACCACTCCCAACAGACCTGTCTATACCACTTCACAAGTCGGAGGCTTAATTACACATGTTCTCTG
GGAAATCGTGGAAATGAGAAAAGAGTTGTGCAATGGCAATTCTGATTGTATGAACAACGATGATGCACTT
GCAGAAAACAATCTGAAACTTCCAGAGATACAAAGAAATGATGGATGCTACCAAACTGGATATAATCAGG
AAATTTGCCTATTGAAAATTTCCTCTGGTCTTCTGGAGTACCATAGCTACCTGGAGTACATGAAGAACAA
CTTAAAAGATAACAAGAAAGACAAAGCCAGAGTCCTTCAGAGAGATACAGAAACTCTAATTCATATCTTC
AACCAAGAGGTAAAAGATTTACATAAAATAGTCCTTCCTACCCCAATTTCCAATGCTCTCCTAACAGATA
AGCTGGAGTCACAGAAGGAGTGGCTAAGGACCAAGACCATCCAATTCATCTTGAAATCACTTGAAGAATT
TCTAAAAGTCACTTTGAGATCTACTCGGCAAACCTAGTGCGTTATGCCTAAGCATATCAGTTTGTGGACA
TTCCTCACTGTGGTCAGAAAATATATCCTGTTGTCAGGTATCTGACTTATGTTGTTCTCTACGAAGAACT
GACAATATGAATGTTGGGACACTATTTTAATTATTTTTAATTTATTGATAATTTAAATAAGTAAACTTTA
AGTTAATTTATGATTGATATTTATTATTTTTATGAAGTGTCACTTGAAATGTTATATGTTATAGTTTTGA
AATGATAACCTAAAAATCTATTTGATATAAATATTCTGTTACCTAGCCAGATGGTTTCTTGGAATGTATA
AGTTTACCTCAATGAATTGCTAATTTAAATATGTTTTTAAAGAAATCTTTGTGATGTATTTTTATAATGT
TTAGACTGTCTTCAAACAAATAAATTATATTATATTT

What is claimed is:

1. A method of making one or more monoclonal antibodies wherein an animal strain prone to develop plasmacyte hyperplasia or plasmacytoma is immunized with an antigen of interest, and antibody producing cells are extracted.

2. The method of claim 1, wherein the antibody producing cells are cultured directly.

3. The method of claim 1, wherein the antibody producing cells are immortalized by cellular fusion.

4. A hybridoma made from an animal by the method in claim 3.

5. The method of claim 1, wherein the antibody producing cells are immortalized by transfection with exogenous DNA.

6. The method of claim 1 wherein the animal contains a transgene overexpressing a mammalian interleukin-6 gene.

7. The method of claim 6 wherein the transgene is human interleukin 6.

8. The method of claim 6 wherein the transgene is murine interleukin 6.


SEQUENCE LISTING (2 sequences)

SEQ ID NO: 1 - DNA, 1201 bp, Artificial Sequence
  Description of artificial sequence note = synthetic construct
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 2 - DNA, 1087 bp, Artificial Sequence
  Description of artificial sequence note = synthetic construct
ccaagaacgatagtcaattccagaaaccgctatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatattt